(19) United States
(12) Patent Application Publication
(10) Pub. No.: US 2002/0161363 A1
(43) Pub. Date: Oct. 31, 2002

(54) Rotational guided electrosurgical electrode loop (GREEL)

(76) Inventors: Steven Fodor, Walden, NY (US); David Kim, Los Angeles, CA (US)

Correspondence Address: Steven Fodor, 2274 Albany Post Rd., Walden, NY, 12586, US

(21) Appl. No.: 09/842,067

(22) Filed: Apr. 26, 2001

(51) Int. Cl. A61B 18/18

(52) U.S. Cl. 606/45

(57) ABSTRACT

Guided Rotational Electrosurgical Electrode Loop characterized by a guide tip, and a curved loop wire to perform a rotational biopsy in bipolar electrosurgical mode

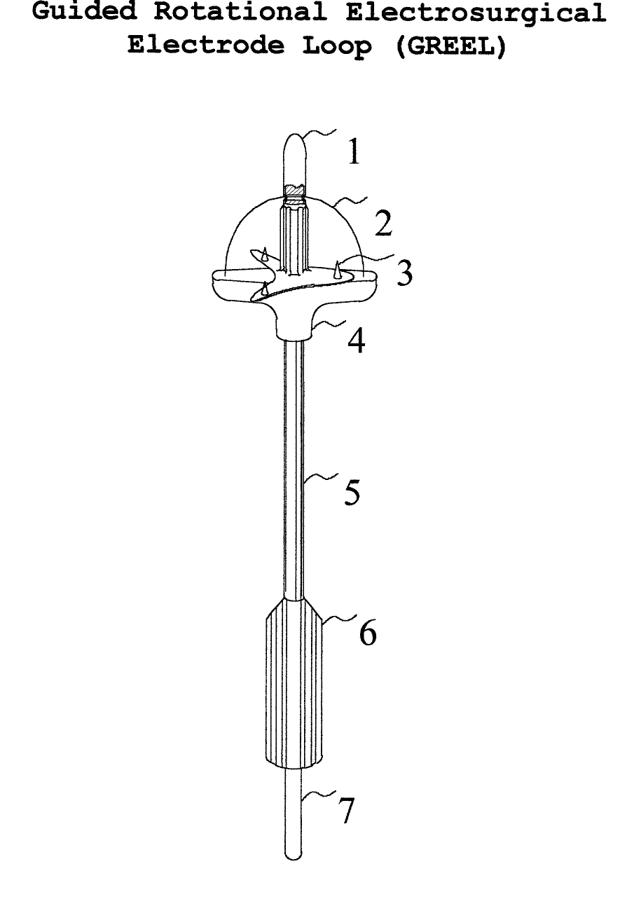

### CROSS REFERENCE TO RELATED APPLICATIONS

U.S. Pat. No. 5,676,663 10/1997 Kim Cone Biopsy Instrument

U.S. Pat. No. 5,324,288 6/1994 Billing et al.

### OTHER PUBLICATIONS

Olsen Electrosurgery, Inc. Catalog 1995 pp. 22

Premier Medical Products, Electrosurgical Products Catalog 2001

Walter Prendiville et al., Large Loop Excision of the Transformation Zone (LLETZ). A new method of the management for women with cervical intraepithelial Neoplasia. British Journal of Obstetric and Gynecology. Sep. 1989. Vol. 96, pp. 1054-1060

### BACKGROUND OF THE INVENTION

This invention relates to a biopsy electrode for the cervical Large Loop Excision of the Transformation Zone (LLETZ). In this procedure a portion of the cervix is resected for diagnosis and treatment of the histologic abnormalities which may lead to a cancer.

The cervical lesion can be removed by various techniques:

a. cold knife cutting, usually complicated by intensive bleeding

b. triangle shaped Olsen or Greenwald type electrosurgical conization electrode, with difficulties to turn the electrode

c. LLETZ electrosurgical electrodes, producing only disorganized fragmented slices of tissue

d. KIM rotational hub cone biopsy electrosurgical electrode (U.S. Pat. No. 5,676,663), with a high degree of safety, but resulting only a straight lined cone

The LLETZ loop electrodes are moved from side to side to remove the cervical tissue. It will produce thin disorganized slices of tissue which will be a poor material for histological diagnosis. The procedure take 5-10 minutes.

As an improvement the KIM electrode is rotated 360 degrees and the monopolar high frequency blend current, along the straight conductive wire, will cut and coagulate the tissue in a straight line at the same time. The current will flow between the monopolar instrument and the large surface dispersive conductive pad. All monopolar techniques require the use of an additional dispersive conductive pad, also described as grounding pads. The KIM electrode will produce a perfectly shaped straight line tissue cone. The KIM electrode has a stabilizing hub with sharp prongs to stabilize the cervical tissue during the rotating cutting.

### SUMMARY OF INVENTION

We have invented a Guided Rotational Electrosurgical Electrode Loop (GREEL) very similar in all other features to the KIM electrode. The instrument holds a symmetrically arranged curved loop around the central guiding tip. The electrosurgical energy is connected to the wire. Once the GREEL is in contact with the tissue, the wire will perform the cutting and coagulation, as current flows away from the wire. To achieve the biopsy, this instrument must be rotated only 180 degrees. The family of the instruments is made in a range of sizes to fit the requirement of surgery. The bipolar model is also possible if the loop is split into two separated sides in the middle. The bipolar procedure is safer than the monopolar one, since there is no need for a grounding pad. There is no danger that burns will occur at the grounding pads. The safety of the bipolar procedure is superior to the monopolar one.

### BRIEF DESCRIPTION OF DRAWINGS

The electrode wire is symmetrically mounted between the central shaft and the middle shoulder. There is central guided tip on the top of the electrode. There is rotating knob on the bottom of the shaft. The exposed bottom terminal is connected to a electrosurgical generator.

We claim:

1. A Guided Rotational Electrosurgical Electrode Loop with a symmetrically arranged curved loop wire, a guide tip, and a freely rotating hub with stabilizing prongs, to perform a loop surgery in monopolar mode.

2. A Guided Rotational Electrosurgical Electrode Loop with symmetrically arranged split and separated curved loop wires, a guide tip, and a freely rotating hub with stabilizing prongs, to perform a loop surgery in bipolar mode.
